(19) United States
(12) Patent Application Publication
(10) Pub. No.: US 2026/0248626 A1
(43) Pub. Date: Aug. 27, 2026

(54) CUSTOMIZED COMPLIANT PROSTHETIC FEET

(71) Applicant: Massachusetts Institute of Technology, Cambridge, MA (US)

(72) Inventors: Charlotte Mery Folinus, Cambridge, MA (US); Amos Winter, Carlisle, MA (US)

(21) Appl. No.: 18/872,661

(22) PCT Filed: Jun. 13, 2023
(86) PCT No.: PCT/US2023/068340

§ 371 Date: Dec. 6, 2024

Related U.S. Application Data
(60) Provisional application No. 63/366,307, filed on Jun. 13, 2022.

(51) Int. Cl. A61F 2/66; A61F 2/50

(52) U.S. Cl. CPC A61F 2/66; A61F 2/5046; A61F 2002/6657

(57) ABSTRACT

Methods of fabricating a compliant prosthetic foot are provided. A method includes optimizing a set of determinants defining a shape of a compliant prosthetic foot. The shape comprises a keel curve, a heel curve, and a forefoot curve, each of the heel curve and the forefoot curve at least partially defined as an offset curve with respect to a representation of an arched ground-facing surface of the compliant prosthetic foot. The optimizing includes minimizing a lower leg trajectory error associated with the set of determinants relative to a target kinematic data set. The method further includes fabricating the compliant prosthetic foot in conformance with the optimized set of determinants.

### RELATED APPLICATION

This application claims the benefit of U.S. Provisional Application No. 63/366,307, filed on Jun. 13, 2022. The entire teachings of the above application are incorporated herein by reference.

### BACKGROUND

Numerous studies have shown that mechanical design of a passive prosthetic foot affects a user's gait. Several metrics are available for assessing quality of a passive prosthetic foot's design, e.g., how well the foot enables the user to replicate normal lower leg kinematics during gait.

One widely-used metric is roll-over geometry, which is defined as a path of a center of pressure during stance phase, as measured in an ankle-knee reference frame. Roll-over geometry offers advantages over other metrics, in that it can be evaluated for typical physiological walking, providing a target design shape, as well as mechanically for prosthetic feet, without inherent variability of human subjects. However, because roll-over geometry is measured in the ankle-knee reference frame, without including any information regarding the orientation of the ankle-knee reference frame relative to the global reference frame, it is possible for two different prosthetic feet to have identical roll-over geometries, yet exhibit very different lower leg kinematics during gait. Therefore, roll-over geometry is insufficient as a design objective.

Another method, called the Lower Leg Trajectory Error (LLTE), quantifies how closely the position of the lower leg segment of a given prosthetic foot is able to replicate target physiological lower leg positions throughout the course of a step. Two degree-of-freedom architectures have effectively proven the concept of prosthetic foot optimization based on LLTE. However, such devices are generally large, heavy and include relatively complex mechanisms.

Methods for calculating shape and size of a passive prosthetic foot for a below-knee amputee are described in Intl. Pub. No. WO 2018/218139, titled “Method for Design and Manufacture of Compliant Prosthetic Foot” (hereinafter referred to as “Olesnavage”), the entire teachings of which are hereby incorporated by reference herein. The Olesnavage passive prosthetic foot (hereinafter referred to as the “Olesnavage foot”) enables the amputee to more closely replicate normal walking motions than was previously possible. Improvements to the Olesnavage foot are described in Intl. Pub. No, WO 2020/247052, titled “Shape Optimization for Prosthetic Feet”, the entire teachings of which are hereby incorporated by reference herein.

There exists a need for improved methods of calculating shape and size of a passive prosthetic foot that can provide for a foot having improved form features.

### SUMMARY

Compliant prosthetic feet and methods of designing and manufacturing compliant prosthetic feet are provided which include improvements to the Olesnavage foot.

A method of fabricating a compliant prosthetic foot includes optimizing a set of determinants defining a shape of a compliant prosthetic foot. The shape comprises a keel curve, a heel curve, and a forefoot curve. Each of the heel curve and the forefoot curve is at least partially defined as an offset curve with respect to a representation of an arched ground-facing surface of the compliant prosthetic foot. The optimizing includes minimizing a lower leg trajectory error associated with the set of determinants relative to a target kinematic data set. The method further includes fabricating the compliant prosthetic foot in conformance with the optimized set of determinants.

Another method of fabricating a compliant prosthetic foot includes combining a compliant mechanism optimization technique that includes a set of determinants for a compliant prosthetic foot with calculation of lower leg trajectory error under a reference loading condition. The set of determinants defines a shape of a compliant prosthetic foot, the shape comprising a keel curve, a heel curve, and a forefoot curve. Each of the heel curve and the forefoot curve is at least partially defined as an offset curve with respect to a representation of an arched ground-facing surface of the compliant prosthetic foot. The method further includes forming an optimized set of determinants of the compliant prosthetic foot that minimizes the lower leg trajectory error relative to a target kinematic data set and fabricating the compliant prosthetic foot in conformance with the optimized set of determinants.

A representation of an arched ground-facing surface can define a surface providing for a heel rise and a foot arch for the compliant prosthetic foot.

A method of fabricating a compliant prosthetic foot includes optimizing a set of determinants defining a shape of a compliant prosthetic foot, the shape being a two-dimensional shape defined by at least one parametric curve. The optimizing includes minimizing a lower leg trajectory error associated with the set of determinants relative to a target kinematic data set. The method further includes generating a three-dimensional shape of the compliant prosthetic foot based on the optimized set of determinants. The three-dimensional shape is defined at least in part by a set of three-dimensional elements of varying widths. The method further includes fabricating the compliant prosthetic foot in conformance with the generated three-dimensional shape.

Another method of fabricating a compliant prosthetic foot includes combining a compliant mechanism optimization technique that includes a set of determinants for a compliant prosthetic foot with calculation of lower leg trajectory error under a reference loading condition. The method further includes forming an optimized set of determinants of the compliant prosthetic foot that minimizes the lower leg trajectory error relative to a target kinematic data set. The set of determinants defines a shape of a compliant prosthetic foot, the shape being a two-dimensional shape defined by at least one parametric curve. The method further includes generating a three-dimensional shape of the compliant prosthetic foot based on the optimized set of determinants. The three-dimensional shape is defined at least in part by a set of three-dimensional elements of varying widths. The method further includes fabricating the compliant prosthetic foot in conformance with the optimized set of determinants.

The varying widths of the three-dimensional elements can vary over a length of the compliant prosthetic foot to complement an interior region of a foot shell. The set of three-dimensional elements can comprise paired three-dimensional elements separated by a gap to provide coronal compliance.

A set of determinants can comprise at least twelve determinants that include at least one geometric parameter at each of six control points of a parametric curve. Optimizing the set of determinants can comprise optimizing the at least twelve determinants for a prosthetic foot that is compliant along its entire length.

Optimizing the set of set of determinants can comprise taking into consideration an intended user's body weight, height, foot size and preferred walking activity.

Optimizing can comprise a compliant mechanism optimization technique that includes a parameterization step for at least one of wide Bezier curve parameters, polynomial interpolation curve parameters, Lagrange function curve parameters. Optimizing can comprise a parameterization step for a topology optimization technique.

The set of determinants of the compliant prosthetic foot can be set by finite element analysis. The target kinematic data set can be a physiological data set obtained from a subject for whom the compliant prosthetic foot is being fabricated, a physiological data set obtained from an able-bodied subject with about the same body size and mass as the subject for whom the compliant prosthetic foot is being fabricated, and/or a physiological data set scaled from an able-bodied subject to adjust for differences in body size and mass compared to the subject for whom the compliant prosthetic foot is being fabricated. The target kinematic data set can be obtained by at least one of simulation, measurement of a subject, measurement from a population of subjects and scaling in magnitude from a subject(s) of a different body size and weight. A target kinetic dataset corresponding to a particular target kinematic dataset can be provided. A target kinetic dataset can be applied to a foot model, which can be used to determine deflection and resulting kinematics. A dataset provided for use in optimization can include a target kinematic dataset, a target kinetic dataset, and a combination thereof. For example, during optimization, a kinematic error relative to the kinematics of the target dataset can be reduced or minimized and/or a kinetic error relative to corresponding kinetics can be reduced or minimized.

A compliant prosthetic foot can be fabricated by at least one method selected from the group consisting of: machining; three-dimensional printing; a layup method; a water jet method; additive fabrication; subtractive fabrication; lamination; composite manufacture; injection molding; carbon fiber fabrication; extrusion; casting; molding; co-molding; carving; and vulcanization. A compliant prosthetic foot can be fabricated of at least one member of the group consisting of: nylon 6/6; carbon fiber; fiber glass; spring steel; titanium; plastic; an alloy of metals; a polymer; a composite; a resin; a thermoset; a thermoplastic; laminate; a rubber; an elastomer; a non-viscoelastic material; a viscoelastic material; and wood.

A compliant prosthetic foot includes a keel, a heel, and a forefoot. The forefoot and heel are shaped according to a parametric curve at least partially defined as an offset curve with respect to a representation of an arched ground-facing surface of the compliant prosthetic foot. The parametric curve(s) providing for the shape of the heel, forefoot, and/or keel can be characterized by a set of parameters defining at least three control circles (e.g., any combination of C₁, C₂, C₃, C₄, C₅, and/or C₆, as shown in FIG. 4A). The forefoot and the keel can be shaped according to a set of three-dimensional elements of varying widths (e.g., in a transverse direction), for example, to conform the foot to a foot shell.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a diagram illustrating an LLTE design framework, shown with force and moment balance analysis in the sagittal plane.

FIG. 2 is a schematic of an improved compliant prosthetic foot.

FIG. 3 is an Ashby-style plot representing two material selection indices: the strain energy density and the relative strength factor metric. All data are normalized to the properties of Nylon 6/6; values≥1 represent higher performance than Nylon 6/6.

FIGS. 4A and 4B illustrate a parametric model for an example foot architecture. FIG. 4A is a diagram of a 2D shape of a prosthetic foot described using Bézier curves defined by control variables Cᵢⱼ, foot ankle height hₐₙₖ, and prosthetic foot length L_{foot}. Twelve independent design variables are shown in black, and nine dependent design variables are shown in grey. FIG. 4B is a diagram of a 3D shape of the prosthetic foot is described using w₁(y), which defines the width of the foot below the fillet, and w₂(x), which defines the width of the foot above the fillet.

FIGS. 5A-5C further illustrate a model for an example foot architecture. FIG. 5A is a diagram illustrating a foot centerline at least partially defined as an offset curve with respect to an arched ground-facing surface of the compliant prosthetic foot. FIG. 5B is a diagram illustrating the foot of FIG. 5A as a point model. FIG. 5C is a diagram illustrating the foot of FIGS. 5A and 5B as defined by a parametric model comprising control points of defined diameters.

FIGS. 6A-6D illustrate a foot-shell-shoe constitutive model. FIG. 6A is a diagram of a prosthetic foot model defined by independent design variables. FIG. 6B is a finite element representation of the foot as frame elements. FIG. 6C is a diagram of a deformed prosthetic foot shape and shell-shoe deformation when a set of reference ground reaction forces (GRFs) are applied at a given center of pressure (CoP). FIG. 6D is a diagram of lower leg orientation and knee position resulting from the deformed prosthetic foot, cosmetic shell, and shoe. The un-deformed shape (light grey) is overlaid with the deformed shape (dark grey) of the prosthetic foot, shell, and shoe.

FIG. 7 is a chart of LLTE-optimal foot designs for users of a range of body sizes. Prosthetic feet optimized for size 27 cm users in a range of body masses and lower leg lengths show different geometries and LLTE scores. In general, LLTE scores are lower for lighter, taller patients.

FIG. 8A is a schematic of an experimental setup for constitutive model testing for prototype foot stiffness.

FIG. 8B is a graph of load-displacement resulting from the testing of FIG. 8A for a prototype prosthetic foot. Measurements were obtained with an Instron machine and predicted using a constitutive structural (FEA) model. Prototypes were tested at the heel (CoPₕₑₑₗ=−30±0.1 mm) and keel (CoPₖₑₑₗ=130±0.1 mm).

FIG. 9A is a schematic of an experimental setup for an International Organization for Standardization (ISO) ultimate static strength testing.

FIG. 9B is a graph of load-displacement curves resulting from the testing of FIG. 9A for a prototype prosthetic foot. The results shown were of a prototype foot designed for an 80 kg, size 27 cm user. Per ISO 10328, the prototype was loaded on the heel at a −15 degree angle and at the keel at a 20 degree angle to a maximum load of 3098 N.

FIG. 10A is a schematic of an experimental setup for an American Orthotic and Prosthetic Association (AOPA) dynamic keel testing.

FIG. 10B is a graph of load-displacement curves resulting from the testing of FIG. 10A for a prototype prosthetic foot. The results shown were of prototype foot designed for a size 27 cm, 80 kg user. Per the AOPA test standards, the prototype was loaded to a peak load of 1230 N at both the heel (−15 deg) and keel (20 deg) platform orientations.

FIG. 11 is a schematic illustrating a three-dimensional model for an example foot architecture.

### DETAILED DESCRIPTION

A description of example embodiments follows.

A general purpose of this invention is to provide for a passive prosthetic foot architecture and design methodology that enable the design and manufacturing of customized prosthetic feet for individual amputees in a way that meets commercial economic, mechanical, and aesthetic objectives.

The provided methods and devices improve and build upon the methods and devices described in International Publication No. WO2018/218139, titled “Methods for Design and Manufacture of Compliant Prosthetic Foot,” and in International Publication No. WO2020/247052, titled “Shape optimization for prosthetic feet,” the entire teachings of which are incorporated herein by reference.

Existing energy storage and return (ESR) prosthetic feet are available in a discrete and low-resolution set of size and stiffness options, which may leave many users with inferior walking performance. Many existing prosthetic foot models are available in ~5 stiffness categories in a given foot length, giving a size-to-size variation in stiffness of approximately 10-15%. This size-to-size stiffness variation is greater than both users' stiffness perception (~5-10%) and repeatability of stiffness preference (~5%) when walking in a variable-stiffness prosthetic foot. This suggests that amputee users may reliably make foot preference selection with a higher resolution than facilitated by existing sizing systems. User stiffness preference may be associated with clinically relevant improvements to gait, such as improved symmetry.

Although the existing size and stiffness categories can work well for many users, they may leave out certain types of users or demographics, who fall outside of height, weight, and mobility norms, particularly women, children, and military personnel. Women often use devices primarily designed for men; as a result, they may be too large or stiff or not designed to accommodate common female footwear. Relative to their body size, children and military personnel may have increased load requirements, requiring prosthetic components capable of withstanding the greater loads associated with higher mobility activities such as walking on varied terrains, participating in recreational sports, or carrying heavy loads. The size and stiffness mismatch between amputees and existing prosthetic feet results in negative clinical and subjective outcomes due to increased gait compensation, long-term injuries, and pain.

Current prosthetic foot design, manufacturing, and provision processes are inherently low-resolution. Providing a greater level of personalization with these techniques is generally either not possible or not commercially viable. Existing prosthetic foot design methods typically require iteration and extensive user-testing. This empirical approach decreases the potential resolution of ESR foot sizes. Existing stiffness categories often informally result from this user-testing, not from a predictive design process. Without a deterministic design methodology, it is not possible to design amputee-specific prosthetic feet. The manufacturing of ESR feet also limits the potential for personalization. ESR foot manufacturing often utilizes expensive composite materials, such as carbon fiber or fiberglass, which require fixed tooling. Increasing the number of size and stiffness options requires investing in additional sets of tooling, directly increasing the manufacturing cost. Prosthetic foot provision processes have co-evolved with the existing resolution of size and stiffness options. Fitting a prosthetic foot can be time-intensive and require many alignment iterations. To compensate for the low resolution of available stiffness options, prosthetists often make small adjustments to foot behavior by iteratively tuning alignment or interchanging reconfigurable foot components. While these components, such as heel wedges and bumpers, may allow for adjustability beyond the coarse sizing systems, they are still low-resolution, and they may have unpredictable, manufacturer-dependent effects. The highly manual fitting process leverages clinical expertise but requires trial-and-error and significant time to converge on an appropriate prosthesis and alignment. Creating a fully customized foot for a patient thus can require many fittings and iterations, which can be cumbersome. Additional per-patient prosthetist time also comes at a cost; the fewer patients seen by a prosthetist, the less revenue generated.

A higher-resolution set of size and stiffness options, designed through amputee-specific personalization, can provide clinical value through improved walking performance and clinical outcome measures. These outcome measures are often related to functional mobility, quality of life, and stability. Restoring mobility and quality of life is often seen as a primary goal of rehabilitation. By reducing gait compensation and risks of long-term injuries, personalized prosthetic devices also have the potential to provide economic value by reducing the overall cost of long-term injuries. This is of particular interest in the United States, where healthcare costs are among the highest in the world, lifetime prosthetic care for a unilateral transtibial amputee costs approximately one million USD, and there is a growing desire to reduce costs through evidence-based prescription practices and amputee-independent metrics.

To be commercially viable, a personalized prosthetic foot can balance the clinical benefits of customization with the functional requirements of the clinical-commercial ecosystem, utilize manufacturing processes which facilitate personalization, and be designed with a form factor and using a design methodology which satisfies these requirements. Despite the promise of personalization, a prosthetic foot which is only personalizable but not also commercially viable may not gain traction as a commercial product. Increasing device personalization also increases the complexity of manufacturing and distribution, but it need not imply full customization and an infinite number of potential foot designs. Infinite customization may be neither clinically necessary nor commercially advantageous.

There is a growing desire to use digital manufacturing processes such as additive manufacturing (AM) to produce patient-specific medical devices; however, existing prosthetic design processes are empirical, iterative, and too slow to facilitate customization. AM can be a promising solution for creating personalized medical devices such as prosthetic feet. Nevertheless, its process capabilities, part-to-part variability, and achievable material properties introduce additional uncertainty.

A manufacturing process capable of producing a high-resolution set of prosthetic foot size and stiffness options can be paired both with a design methodology which facilitates personalization and with an appropriate embodiment of foot geometry. The lower leg trajectory error (LLTE) design framework can provide a quantitative methodology for prosthetic foot design, facilitating the customization of prosthetic feet based on an individual's body size and activity level and filling a knowledge gap in prosthetic foot design theory. In prior gait testing, LLTE feet were customized for users with a range of body sizes, and the LLTE-designed prototype feet showed similar or better biomechanical performance and subjective ratings when compared with commercially available, carbon fiber ESR prosthetic feet. The LLTE methodology is also capable of producing prosthetic feet which meet commercial strength standards such as ISO 10328 and which survive extended use in rugged conditions.

A diagram of an LLTE design framework 100 is shown in FIG. 1, with force 102 and moment 104 balance shown in the sagittal plane. A position and orientation of the lower leg segment 110 can be defined by a position of the knee (xₖₙₑₑ, yₖₙₑₑ) and an angle of the lower leg segment (θ_{LL}). These coordinates can be calculated from a deformed shape of the prosthetic foot 112 under prescribed loading conditions (GRFₓ and GRF_{y} at a specific center of pressure (CoP)).

The LLTE framework can connect a mechanical design of a prosthetic foot with its anticipated biomechanical performance, providing a deterministic method for tuning the geometry and stiffness of a prosthetic foot to yield a desired biomechanical response. The LLTE framework can use a constitutive, or finite element, model of the prosthetic foot to compute its deformation and the resulting lower leg trajectory when target reference loads (e.g., ground reaction forces applied at the corresponding centers of pressure) are applied. The LLTE metric quantifies the foot's biomechanical performance throughout stance by computing the deviation (i.e., error) between the prosthetic side lower leg trajectory and the target reference lower leg trajectory. The LLTE metric can be defined as follows, where the superscripts “model” and “ref” correspond to the values computed from the constitutive model and those from the reference data set, respectively:

$$LLTE = {[ \frac{1}{N} {\sum }_{n = 1}^{N} \{ \begin{matrix}{( \frac{{x}_{knee , n}^{model} - {x}_{knee , n}^{ref}}{{L}_{lowerieg}} )}^{2} \\ + {( \frac{{y}_{knee , n}^{model} - {y}_{knee , n}^{ref}}{{L}_{lowerieg}} )}^{2} \\ + {( \frac{{θ}_{LL , n}^{model} - {θ}_{LL , n}^{ref}}{atan ( \frac{{L}_{lowerieg}}{{L}_{foot}} )} )}^{2}\end{matrix} \} ]}^{1 / 2} \qquad ( 1 )$$

For each individual frame n (time instance of throughout stance) of N total frames, the knee coordinates and lower leg orientation can be calculated. The deviations from each reference variable can normalized by the lower leg length L_{lowerleg} and a tan(L_{lowerleg}/L_{foot}). The LLTE metric can be incorporated in an optimization to systematically adjust the mechanical properties (e.g., geometry and stiffness) of a prosthetic foot, resulting in foot designs which minimize the LLTE value. The lower the LLTE value, the more closely the prosthetic foot enables replication of the target walking pattern; the LLTE-optimal design is that which best enables this replication. Although LLTE-designed feet have used Nylon 6/6, a relatively low-cost material, the systematic and quantitative LLTE design results in feet with similar biomechanical performance and subjective evaluation to existing commercial feet, which are made of costlier composite materials such as carbon fiber or fiberglass.

Prior prototypes of LLTE-designed prosthetic feet had relatively simple form factors which could be fabricated using, for example, a waterjet and two-axis milling machine. To minimize gait variation due to footwear during biomechanical testing, these prototypes were designed to be worn overground. As a result, the prototype devices did not fit within commercial foot shells, which is the typical form factor for ESR feet. Additionally, the prototype foot design did not consider the additional compliance introduced by a foot shell or shoe.

Methods and devices are described which can provide for LLTE-designed prosthetic feet with improved mechanical features. Material and manufacturing features for producing such prosthetic feed are provided. A mechanically-validated foot form that can be designed in a high-resolution set of size and stiffness options, allowing for personalization while meeting core design requirements, is provided.

Compliant prosthetic feet can include features providing personalization, performance, and rapid manufacturing, while also satisfying typical insurance requirements. An example list of design parameters and specifications is shown in Table 1. These design parameters can be included, in any combination, with LLTE and curve parameters toward the design of a compliant prosthetic foot.

TABLE 1
Design parameters for a high-performance, commercially
viable, and personalized prosthetic foot

| | Measurable Parameter and |
|---|---|
| Feature | Example Values/Characteristics |
| Customizable for patient | Heel height - 10 mm |
| at point-of-purchase | Foot size - size 18-30 cm |
| | User weight - 45-136 kg |
| | Build height - 140+ mm (with adaptor) |
| | Activity level - low/high options |
| Rapidly providable | Takes ≤2 business days from incoming |
| | order to outgoing shipment |
| High performance | Meets Freedom Highlander in subjective |
| | rating and clinical outcomes |
| Lightweight | Weighs ≤0.6 kg without cosmetic foot |
| | shell (in size 27 cm) |
| Passive | Does not use hydraulic or powered components |
| Compatible with | Passes ISO 10328/22675 tests |
| L5981 standards | Passes AOPA dynamic heel and keel tests |
| Compatible with | Can be attached via standard pyramid adapter |
| US prostheses | Is worn with existing commercial foot shell |

Personalization, or customization, is a valuable feature for a prosthetic foot. Some existing prosthetic feet provide a limited amount of post-purchase adjustability by mixing-and-matching reconfigurable modular components such as heel wedges or bumpers, and some prosthetists value this adjustability. This practice may stem from the limited resolution of existing size and stiffness options, and it may not reflect a desire or need for post-purchase adjustability of a personalized prosthetic foot. The compliant prosthetic feet provided can enable point-of-purchase customization, or personalization that can occur when a prosthetist orders a device.

The foot size and user weight ranges were based off of both existing commercial feet and US population data. A prosthetic foot can be designed for K3 populations, which can drive the heel height and build height requirements. A K-level is an indicator of an activity level. ESR feet that facilitate a high level of activity are typically K3 to K4. Prosthetic feet for any K-level (e.g., K2, K3, K4) can be designed by the provided methods. To accommodate the heel rise in many shoes, cosmetic foot shells and the prosthetic feet that are worn within them have an elevated heel. In most K3 feet, this heel height is typically 10 mm, or ⅜″. A prosthetic foot's build height involves a tradeoff: a lower foot build height (shorter foot) accommodates a greater range of residual limb lengths, but a larger build height allows the foot to store and return more energy. To balance this tradeoff, a minimum build height of 140 mm can be selected, which is similar to the build height of the 6″ (152 mm) Fillauer All Pro [1], a device which suppliers sometimes use as a benchmark for the height of new products.

The adjustments a prosthetist can make in ordering a personalized prosthetic foot can be clinically meaningful. The LLTE design framework can allow clinicians to fine tune the “activity level” of a patient by specifying the walking activities for which the foot is designed, such as walking up ramps or at walking quickly on flat ground. Although the capability to quantitatively design a prosthetic foot for a specific activity is available, an infinite resolution of activity level options is likely unnecessary. Beyond a certain granularity, adjustments to the target user profile may be neither repeatable nor clinically meaningful.

To make sense in existing clinical prosthetist workflow, rapid provision is also desirable. The fulfillment time of less than two business days from order receipt is based off of the lead time from order receipt to shipment of current products. While prosthetists may be willing to accept a slightly longer lead time in exchange for a more personalized prosthetic foot, increasing the fulfillment time beyond two days may disrupt their workflow, proving a deterrence and a barrier to adoption.

For prosthetists to change their existing behaviors and prescribe a new prosthetic foot, the new product can provide for equivalent or better performance than existing products. The Freedom Highlander can be selected as a commercial benchmark due to its widespread acceptance and provision in the clinical community.

The prosthetic foot can be designed for distribution in the US under L5981, a category of ESR prosthetic feet commonly prescribed to K3/4 amputees. Traditionally, passing the AOPA dynamic heel and keel tests has been considered criteria for classification as an L5981 product. To pass these tests, a prosthetic foot must exceed a given amount of deformation and energy return efficiency in representative heel and keel loading scenarios. To demonstrate strength and durability, particularly for insurance providers, prosthetic feet typically must survive both static and cyclic load testing according to ISO 10328 or 22675. Satisfying the ISO strength and durability tests requires that a prosthetic foot be strong enough to withstand both high single-cycle loads (static proof and ultimate strength) and lower magnitude multi-cycle loads (fatigue) without plastic deformation.

Compatibility with the common ESR prosthetic foot form factors in the US can be necessary for adoption by prosthetists and acceptance by amputee patients. To best utilize prosthetists' clinical experience and avoid retraining with an additional mounting system, a personalized prosthetic foot can attach to the rest of the prosthesis using a standard pyramid adapter. Many amputees desire a physiological prosthetic foot appearance; to achieve this appearance with the walking performance provided by existing ESR feet, L5981 feet are commonly worn within a commercial foot shell, which is a thin, removable foam enclosure which looks like a physiological foot and houses the prosthetic foot. Rather than design a new foot shell for a prosthetic foot, the foot can be adapted to fit within an existing shell.

A prosthetic foot can be designed to be low-cost to manufacture. To meet this need, an upper cost threshold of ~5% of the minimum reimbursement for the L5981 code can be met. Reimbursement amounts vary geographically across the United States and fluctuate over time. A low manufacturing cost may not reduce insurance reimbursement costs, but it can reduce purchase costs to clinics.

ESR prosthetic feet are able to store and return energy with each step, and they are typically strong enough to withstand years of daily use. Instead of focusing on individual material properties such as elastic modulus, yield strength, or ultimate strength, material performance indices can be utilized which directly relate to the energy storage and strength requirements of the ISO and AOPA tests.

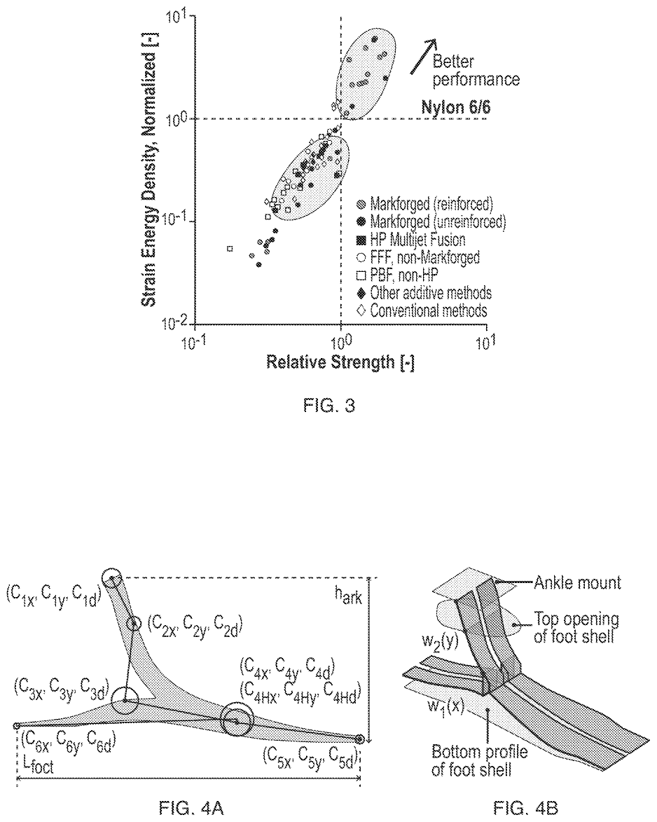

The ability of a prosthetic foot to satisfy these requirements can relate to both its material and geometry. A prosthetic foot's material performance was quantified through two performance indices, the strain energy density and the relative strength factor (FIG. 3). Material properties were compared to Nylon 6/6, a high performance and relatively inexpensive thermoplastic which has been used in prior work. Values for both performance indices are divided by the values for Nylon 6/6. High scores correspond to high performing materials, with values greater than one indicating that a material outperforms Nylon 6/6 (tensile modulus E=2.49 GPa, tensile yield stress σ_{y}=73.7 MPa, poisson ratio ν=0.6, and density ρ=1130 kg/m3). Properties are given from manufacturer-supplied tensile data or tensile data in academic publications. Although bending is the primary deformation mechanism in ESR prosthetic feet, flexural data is less commonly available, and material properties for tensile and flexural testing are well-correlated for the polymers and composites considered here.

The first performance index, the strain energy density represents a material's potential for recoverable energy storage. The strain energy density use for a material with elastic modulus E, yield stress σ_{y}, and yield strain ϵ_{y} is given as follows, with units of energy per unit volume (J/m³):

$${u}_{SE} = {σ}_{y} {ϵ}_{y} = \frac{{σ}_{y}^{2}}{E} \qquad ( 2 )$$

A material with a high strain energy density is able to store a large amount of energy per unit volume before reaching the yield stress. For materials which fail by brittle fracture instead of plastic yield, σ_{y} and ϵ_{y} can be replaced with fracture stress of and strain ϵ_{f}, respectively. A material with a normalized score greater than one (FIG. 3) can elastically store and return more energy per unit volume than Nylon 6/6.

While the strain energy density metric quantifies material performance, it does not consider the base geometry which will be used. The relative strength factor quantifies both material and geometric performance; it represents how much a design can change to meet safety factor constraints. A relative strength factor can be calculated by comparing the strength of two cantilevered beams with the same equivalent bending stiffness, (EI)_{eff}, but different materials and cross-section geometries, where Eᵢ and Iᵢ represent the elastic modulus and second moment area for beam I with width wᵢ and thickness tᵢ:

$${( EI )}_{eff} = {( EI )}_{i} = \frac{1}{12} {E}_{i} {w}_{i} {t}_{i}^{3} , \qquad ( 3 )$$

Solving Eqn. 3 for tᵢ in terms of (EI)_{eff} gives:

$${t}_{i} = {( \frac{12 {( EI )}_{eff}}{{w}_{i} {E}_{i}} )}^{1 / 3} \qquad ( 4 )$$

To bend the same amount, a beam of a softer material (lower Eᵢ) must be thicker (higher tᵢ), if both beams have the same width w. The maximum stress and safety factor associated with a tip loaded, cantilever beam-based design can be given as:

$${σ}_{max} = \frac{{FL}_{{y}_{max}}}{EI} = \frac{FL}{{( EI )}_{eff}} ( \frac{{t}_{i}}{2} ) ∝ {E}_{i}^{2 / 3} \qquad ( 5 )$$

Comparing the maximum stress associated with loading a design (Eqn. 5) to its yield strength gives a safety factor SF and relative strength factor γ_{SF}:

$$SF = \frac{{σ}_{y}}{{σ}_{max}} ∝ \frac{{σ}_{y}}{{E}_{i}^{2 / 3}} \qquad ( 6 )$$

$${γ}_{SF} = \frac{{σ}_{y}}{{E}_{i}^{2 / 3}} \qquad ( 7 )$$

This analysis considers cantilever beams with a solid cross section (I=( 1/12)wt³), but the relative strength factor calculations can be extended to beams with more complex cross sections such as leaf springs, I-beams, or multi-material, composite beams. A combination of material and geometry with a normalized relative strength score below one is weaker than Nylon 6/6. This means material can be added to meet the same stress or safety factor constraints. Adding material creates a stiffer prosthetic foot, which limits walking performance.

Extruded Nylon 6/6 was found to outperform additively manufactured (AM) polymer materials (FIG. 3). Among these polymers, the Nylon 11- and 12-based materials from HP were the highest performing. Of all AM materials considered, only the Nylon-reinforced composites from Markforged were found to potentially outperform extruded Nylon 6/6; however, their behavior is more uncertain due to a lack of consensus on how the location and quantity of fiber reinforcement impacts stiffness and part failure, limited research of the fatigue behavior of printed composites, and significant part-to-part variability in mechanical properties. Cast urethanes offer similar performance to extruded Nylon 6/6, but casting has a limited ability to accommodate the geometric features needed for a commercially-appropriate prosthetic foot form factor. Additionally, casting requires fixed tooling, which is undesirable for manufacturing a personalized product at scale. Comparing materials based on performance metrics revealed three primary candidates: extruded, machined Nylon 6/6; Nylon-based composites from Markforged; and Nylon 11 and 12 from HP.

While the above materials were tested, it should be understood that a compliant prosthetic foot can be made from materials other than those tested. A compliant prosthetic foot can be fabricated of a material comprising, for example, nylon (e.g., nylon 6/6, nylon 11, nylon 12), carbon fiber, fiber glass, spring steel, titanium, plastic, an alloy of metals, a polymer (e.g., AM polymers), a composite, a resin, a thermoset, a thermoplastic, laminate, a rubber, an elastomer, a non-viscoelastic material, a viscoelastic material, and wood.

The manufacturing rate, or the speed at which parts can be produced, can directly connect to design requirements and can impact the overall manufacturing cost. A manufacturing process which takes more than two business days to produce an individual prosthetic foot does not satisfy the proposed requirement and may not be not viable. Compared with a faster and higher throughput process, a slower manufacturing rate requires purchasing additional equipment to meet the same production volume.

The overall manufacturing time was estimated using a representative early-stage model of the prosthetic foot design for three processes: CNC machining of Nylon 6/6, Markforged's continuous filament fabrication, and HP's multijet fusion. CNC machining time was estimated using a material removal rate calculation. A material removal rate MRR of 26 cm³/min, and a total volume of material Vt of 1200 cm³ gives an estimated machining time t=Vₜ/MRR=46 minutes, or 0.77 hr/foot. For a single prosthetic foot with a base material of Nylon White (solid infill) reinforced with Kevlar (two concentric fiber rings, 150 reinforced layers), Markforged's Eiger software predicted a print time of 48 hours on either its desktop or industrial machines. For a fully nested build of 25 parts with same part file and a base material of Nylon 12, HP's SmartStream software predicted a build time of eight hours and cooling time of eight hours, for a total of 16 hours, giving an average of 0.64 hr/foot. Both HP MJF and CNC machining can satisfy a≤2 business day time requirement to design and manufacture a prosthetic foot, but Markforged did not meet this requirement.

Manufacturing quality can be described in many ways, from performance- and durability-related metrics to factors such as reliability, aesthetics, and perceived quality. Process reliability can also be a consideration. Creating high resolution and personalized prosthetic feet centers on the value proposition that user-specific designs could be superior to existing ESR feet, which are available in a low resolution of size and stiffness categories. A high variability manufacturing process may not provide this value, as it will not consistently result in prosthetic feet with the target static or fatigue behavior.

The outputs of manufacturing processes are subject to variability, which might be due to variations in the raw material or inconsistent machine behaviors. This results in variations in part stiffness due to changes in material properties and manufactured part geometry, which can be modeled by representing the prosthetic foot structure as an Euler Bernoulli beam. A beam with Young's Modulus E, moment of inertia I, and length L has an effective stiffness k=3EI/L³, or k∝E. Variation in stiffness is directly proportional to variation in Young's Modulus; a 5% change in modulus results in a 5% change in stiffness. Variation in part-to-part elastic modulus may depend on the process parameters and part configuration. For Markforged CFF, one study reports part-to-part variation of flexural modulus of 3.5%-7.9%, while another reports variation ranging from 1.6% to 22.2%. For HP MJF, this variation has been reported as 0.7% to 4.5%, depending on the part orientation. The variation in extruded Nylon 6/6 is 1.2%, which is similar or better than HP, and lower than the variation in Markforged CFF material properties. The part-to-part variation of Markforged CFF parts exceeds the amputee-perceivable difference in stiffness.

While geometric repeatability is generally good for Markforged CFF, HP MJF, and CNC machining, these tolerances can become significant for the part geometries considered for a compliant prosthetic foot. Thermal strain during part cooling in both the CFF and MJF processes can lead to thermal distortion, which can manifest as part contraction and warping. While this effect is small for relatively thin and small parts, such as those used for the materials tests common in literature, thermal strain is much greater for thick, large parts such as the prosthetic foot geometries considered. Dimensional tolerance in CNC machining is often specified to ISO 2768 medium tolerance, which provides a maximum permissible deviation based on the nominal part dimension, such as +0.2 mm for dimensions between 6 mm and 30 mm. This corresponds to an overall change in stiffness ranging from 0.2/30 mm/mm to 0.2/60 mm/mm, or 0.7%-3.3% change in stiffness due to geometric variation, which is below the amputee-perceivable difference in stiffness.

With current manufacturing capabilities, CNC machining of Nylon 6/6 is a process-material combination which can provide for core design requirements (Table 1). Nylon 6/6 is a high-performance material which can elastically store and return large amounts of energy during the stance phase of walking, and CNC machining is the process which best satisfies manufacturing rate, cost, and quality requirements. Although additive manufacturing is often seen as the hallmark process for customized products, current commercially-available materials and processes may not match the material performance, manufacturing rate, cost, and quality of CNC machining of Nylon 6/6. While Markforged composites could potentially provide higher material performance, the process does not meet requirements for manufacturing rate, cost, or quality. HP's Nylon 11- and 12-based materials do not perform well enough in a prosthetic foot, but the process satisfies rate, cost, and quality requirements. Nylon 6/6 is a high-performance material, and CNC machining can satisfy rate, cost, and quality requirements, even when prosthetic feet are machined as one-of-a kind, unique parts.

The prosthetic feet previously created using the LLTE design framework were designed as experimental prototypes. Improvements to the methods and devices provided by prior LLTE design frameworks can provide for several advantages. For example, a set of determinants defining a shape of a compliant prosthetic foot can provide for variable widths in the frontal plane and/or an elevated heel. Such additional features can provide for an ability to conform the prosthetic foot to fit within a cosmetic foot shell and with shoes. In another example, a set of determinants can be modified to provide for a split keel to provide additional coronal compliance. A split keel can enable improved adaptability on uneven ground and may be preferred by patients. A constitutive model and LLTE calculations can also be modified to include an added compliance of the foot shell and shoe.

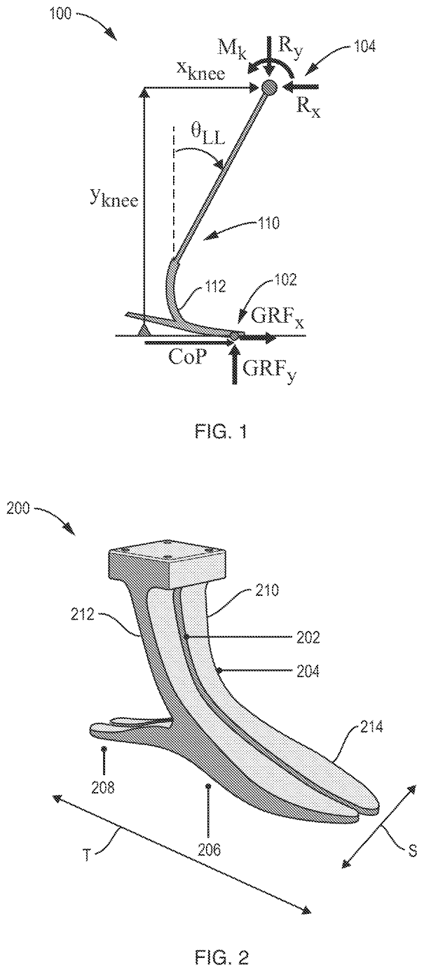

An example of an improved compliant prosthetic foot is shown in FIG. 2. The prosthetic foot 200 includes a body 210 that defines a keel split 202. At a keel 212 of the foot 200, the body 210 further defines a tapered portion 204 at a location at which the keel 212 transitions into a forefoot 214. The body 210 can further define an arch 206 and a heel 208 which is elevated relative to the forefoot 214 with respect to a ground surface. As is visible in FIG. 2, the body 210 has a profile which varies in three-dimensions, as opposed to two-dimensional variations as provided in the prior art approaches. In particular, both a transverse silhouette T and a sagittal silhouette S of the foot 200 can be defined based a set of determinants, which can be iterated or optimized based on LLTE. The determinants can define curves for each of the keel, heel, and forefoot of the foot. Widths of the foot (e.g., in the sagittal plane) can be defined to vary over a length (e.g., in the transverse plane) to provide for tapering, heel rise, and/or split keel features.

A prosthetic foot architecture can be modeled as a 3D compliant structure using wide Bézier curves and boundary curves (e.g., as can be defined by a cosmetic foot shell). Traditional topology synthesis and optimization methods often require a large number (thousands) of design variables to converge to optimal results with high stress concentrations, and suffer with manufacturability due to small feature sizes or checkerboarding patterns. Compared with these approaches, parametric representations such as Bézier curves or B-splines can more efficiently handle stress and manufacturing constraints. A wide Bézier curve is a parametric curve whose shape is defined by a series of control circles. The positions of the control circles describe the shape of the curve, and the circles' diameters define the thickness of the curve. Together, the curves' shapes and thicknesses can define the foot's stiffness and geometry. While wide Bézier curve parameters are described in examples, other curves and parameters for such curves can be used, such as, for example, polynomial interpolation curve parameters, Lagrange function curve parameters, and cubic curve parameters.

In prior work, a 2-D shape and thickness of the prosthetic foot using a series of simpler Bézier curves joined end-to-end, known as a composite wide Bézier curve, is described. In both the previous foot design and the provided, improved foot architecture, three wide Bézier curves are used to describe the 2-D shape of the foot. A main keel portion can be represented as a cubic wide Bézier curve with control circles C1 to C4, a forefoot portion can be represented as a linear segment with control circles C4 and C5, and a heel portion can be described as a linear segment with control circles C4 and C6. Unlike the previous, flat-footed design, where the shapes of the heel and forefoot segments were defined as linear projections from a horizontal line representing the bottom of the foot, these segments can be represented as (linear) offset curves relative to a curved bottom of the foot. Here, a bottom of the foot can be represented as a Bézier curve defined by the length of the foot L_{foot}, the height of heel rise hₕₑₑₗ, and the height of the arch h_{arch}.

A 2-D foot shape can be represented with seven control circles, each of which is defined by three variables (x- and y-positions and diameter), as well as hₕₑₑₗ and h_{arch}, giving a total of 23 degrees of freedom. Of these, six can be defined by a patient's foot length and residuum length, three can be coupled to another design variable, and two can be removed by fixing a center of control circle C₁ as the reference ankle-foot origin. This provides 12 independent design variables which can be tuned to describe a shape of the prosthetic foot (FIG. 4A, see bolded control variables).

A 3-D shape of the foot can be defined by varying element widths along the shape of the foot. These widths can be defined from 2-D cross sections of an interior of a cosmetic foot shell, at the bottom of the foot and the top opening of the foot shell, and by fixing ta width at the top of the foot to wₐₙₖₗₑ to a value (e.g., 52 mm, the width of a four-hole pyramid adapter). The width of the foot at each 2-D location (x, y) can be defined according to a piecewise definition, where y_{fillet} represents a vertical position where the heel curve intersects the main keel curve, w₁(x) and w₂(y) represent boundary curves determined by the shape of the cosmetic foot shell, and wₛₗₒₜ represents a width of the coronal plane slot in the prosthetic foot:

$${w}_{foot} ( x , y ) = \{ \begin{matrix}{w}_{1} ( x ) - {w}_{slot} & y \leq {y}_{fillet} \\ {w}_{2} ( y ) - {w}_{slot} & y > {y}_{fillet} ,\end{matrix} \qquad ( 9 )$$

Below the fillet, a bottom profile of the foot shell can define the width of the prosthetic foot. The variable w₁(x) represents a maximum symmetric width of the inside of the foot shell and is a function of x, the horizontal location along the bottom of the foot. Above the fillet, the side of the prosthetic foot can be constrained to intersect the bottom profile of the foot shell, the top opening of the foot shell, and the ankle mount. The variable w₂(y) can be defined by a cubic interpolating spline through (y_{fillet}, w₁(x_{fillet})), (yₛₕₑₗₗ, wₛₕₑₗₗ(xₛₕₑₗₗ)), and (yₐₙₖₗₑ, wₐₙₖₗₑ). The variable wₛₕₑₗₗ(xₛₕₑₗₗ) can be computed as the width of the top opening of the shell at the intersection of the Bézier curve (xₛₕₑₗₗ, yₛₕₑₗₗ). This representation of the side profile can both define the 3-D foot width and implicitly constrain the 2-D foot shape to fit within the cosmetic foot shell; designs which do not pass through the top opening of the foot shell can be considered geometrically invalid. The foot designs presented here were designed to fit within the Venture foot shell from College Park; however, any foot shell can be used after defining appropriate transverse plane profiles for the top opening and plantar (bottom) surface (FIG. 4B).

Representing the 2-D shape of the foot design using wide Bezier curves and then determining the 3-D shape enables a variety of prosthetic foot designs with varying stiffness and geometry with a low number of design variables.

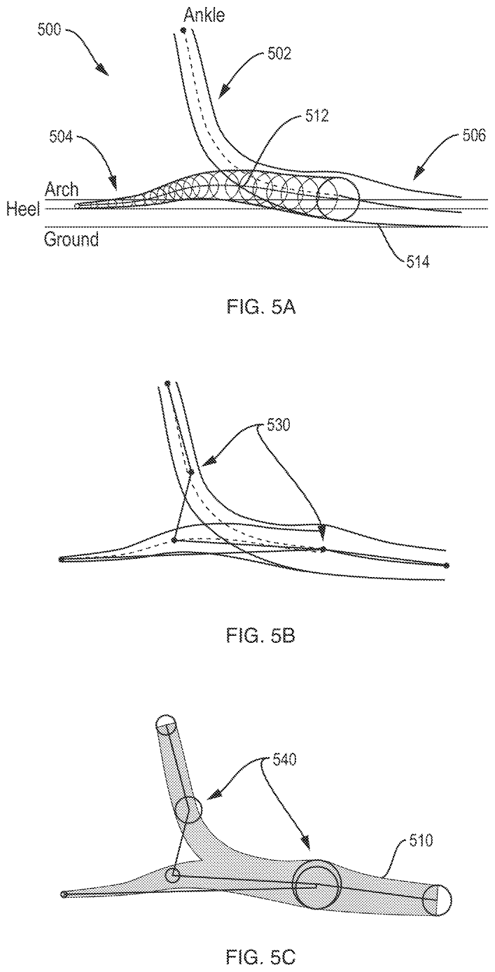

As further illustrated in FIGS. 5A-5C, a model 500 of a compliant prosthetic foot can comprise a keel curve 502, a heel curve 504, and a forefoot curve 506. A centerline 512 of the foot can be at least partially defined as an offset curve with respect to an arched, ground-facing surface 514 of the compliant prosthetic foot. The centerline can be of the heel curve, the keel curve, and/or the forefoot curve. As illustrated, the centerline is of the heel curve and the forefoot curve. Locations along the curves defining the prosthetic model are represented as points 530 in FIG. 5B. As further illustrated in FIG. 5C, control circles 540 can be provided at such locations. Parameters defining the control circles 540 can provide for a set of determinants for defining a body 510 of the prosthetic foot. The determinants can be iterated or optimized based on a LLTE framework to provide for the design of a personalized, compliant prosthetic foot for a subject.

As illustrated in FIG. 11, a model 600 of a compliant prosthetic foot can comprise three-dimensional elements 610 of varying widths. For example, a two-dimensional model of a prosthetic foot (as shown in the transverse plane sectional views of FIGS. 5A-5C) can be used to generate a three-dimensional shape for a prosthetic foot comprising elements having varying widths in the sagittal plane, as shown in FIG. 11. The model can include a pairing of three-dimensional elements (e.g., elements 612a, 612b) separated by a gap 614 to provide compliance in a coronal plane.

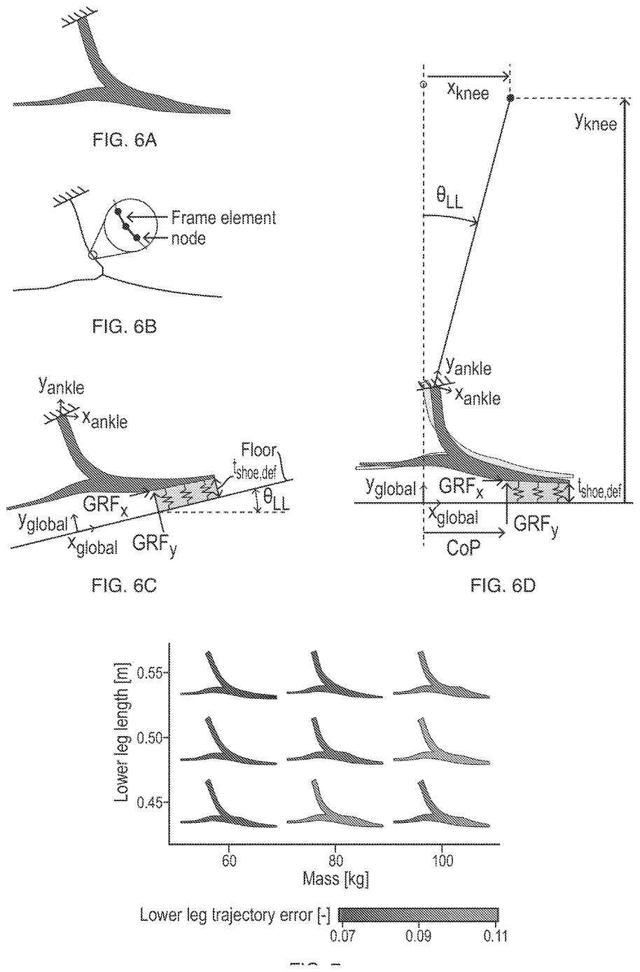

Prosthetic foot design using the LLTE framework can include a model to predict foot deformation and stress distribution under the anticipated loading. The foot deformation can be used to calculate the LLTE metric (Eqn. 1) for a given design, which can be used as the objective function to iterate and, optionally, optimize the prosthetic foot's geometry and stiffness. The stress distribution can be used to compute the effective safety factor, which can act as a constraint to ensure the strength and durability of optimized foot designs. The single-keel constitutive model can thus be extended to accommodate more complex foot architecture and to include the compliance of the cosmetic foot shell and shoe (FIGS. 6A-6D). Footwear can introduce additional compliance to and can alter the mechanical behavior of a prosthesis; without incorporating this behavior, a prosthetic foot may be overly compliant when worn with shoes. A constitutive model can include a 2D finite element model based on frame elements.

The finite element model can be built in, for example, MATLAB (Mathworks, Natick, MA), which can facilitate simple and computationally inexpensive analysis, compared with commercially available structural analysis tools. Although the prosthetic foot has 3-D geometry, the 2-D frame element representation facilitates a significant cost savings without sacrificing accuracy. A 2-D frame element has six degrees of freedom (DOF), compared with the 12 DOF of a 3-D frame element. The mesh size (e.g., 300 elements) can be chosen to ensure a minimal error (1%) in estimated element stiffness due to the 2-D element simplification. While focusing on the kinematics and kinetics of the sagittal plane simplifies the reference walking patterns, this is an appropriate simplification; more than 90% of the work done by the ankle during walking occurs in the sagittal plane. Structural analysis occurs in the ankle reference frame, with the origin and a fixed-fixed boundary condition located at the prosthesis ankle, defined as the center of the C₁ control circle.

To compute a knee position and lower leg orientation when the foot is worn within a cosmetic foot shell and shoe, the deformed thickness of these components can be included in the calculations by modeling their combined stiffness as a simplified linear spring. The stiffness of the spring can be determined using the approximate width, thickness, and length of engagement of the shoe at the heel and toe; and using the material properties of ethyl vinyl acetate (EVA) and polyurethane (PU) foam, which are common foams for shoe midsoles and cosmetic foot shells, respectively.

The provided compliant prosthetic feet can comprise a unitary body. For example, the prosthetic foot can be a single-part device.

A “compliant mechanism optimization technique” is a means of searching for, identifying and designing a structure for a targeted deflection under a given load. A genetic algorithm, or other optimization technique, may be used to determine the optimized set of determinants.

A compliant mechanism optimization technique can include a parameterization step for a topology optimization technique. A “topology optimization technique” can be or include any algorithmic process that can discern an efficient design based on a set of constraints or characteristics. Topology optimization techniques typically include an analysis of a number of connected components/boundaries belonging to a domain. Parameterization steps for a topology optimization technique for a compliant prosthetic foot can include parameterization of, for example, wide Bezier curve parameters, polynomial interpolation curve parameters, and Lagrange function curve parameters, among others.

A “compliant prosthetic foot” is a foot that deforms under load.

A “reference loading condition” is a targeted or anticipated loading that a foot could experience.

An “optimized set of determinants” is a set of variables describing size, form, shape, material and/or structure of a prosthetic foot in a configuration to provide a targeted deflection under a given load.

As used herein, “optimizing” a set of determinants means determining values for such determinants to provide for a prosthetic foot that meets design objectives and does not strictly require arriving at optimal determinants for minimizing a lower leg trajectory error. An optimized set of determinants can, for example, provide reducing a LLTE to a suitable threshold.

The design methodology provided is based on the Lower Leg Trajectory Error (LLTE) design framework. This design framework involves predicting the lower leg trajectory of an amputee user walking in a specific prosthetic foot, comparing this trajectory to able-bodied walking patterns, and optimizing the stiffness and geometry of the prosthetic foot to minimize the difference in the two trajectories.

Existing prosthetic feet are limited by their discrete, low-resolution set of size and stiffness options, which are a result of the design and manufacturing processes which are inherently low resolution. The novel foot architecture described combined with LLTE design theory allows the design of customized prosthetic feet which can be manufactured and provided through existing clinical distribution networks.

Using the provided design framework, a custom prosthetic foot for an amputee based on their body size and activity level can be quantitatively designed. This facilitates a higher-resolution set of size and stiffness options, which can facilitate improved walking performance for amputee users.

While existing commercial ESR feet work well for many users, they are made with iterative design processes and manufacturing processes that prohibit a significant increase in the number of size and stiffness options; this leaves many users with limited walking performance and higher rates of long term overuse injuries. Many prosthetic foot design methods in academia are also iterative and empirical, and the feet may be designed with a focus on theory, not on real-world techno-economic requirements. Provided are methods for designing and manufacturing a high-resolution, amputee-specific personalized prosthetic foot that fits within the exiting clinical-commercial ecosystem. A set of design parameters included economic, mechanical, and aesthetic design requirements (Table 1). To select an appropriate material and manufacturing method, material properties as well as the rate, cost, and quality of manufacturing processes were compared. Using this analysis and the core design objectives, a manufacturing process, CNC machining, and material, Nylon 6/6, were selected. Also provided is a novel foot form which can satisfy the design objectives, can be CNC machined from Nylon 6/6, and can be quantitatively customized through amputee-specific personalization. This foot form can be designed for a variety of user body sizes by optimizing the parametric variables which define its shape.

Experiments were conducted to validate that the foot form fits within a commercial foot shell, mechanically behaves as predicted, and passes the ISO ultimate strength test, the AOPA dynamic heel test, and the AOPA dynamic keel test, as further described in the Exemplification section herein.

A higher level of customization in prosthetic foot design has the potential to provide both clinical and economic benefits. In prior testing of LLTE-designed feet, it was found that users better replicated able-bodied walking patterns, had improved clinical outcomes such as roll-over geometries, trunk sway, prosthetic energy return, and peak push-off power, and preferred their LLTE-optimal feet, compared with a commercially-available control prosthetic foot and stiffness variants of their LLTE-optimal design. Improving amputee quality of life and decreasing risk of long-term injuries can provide economic value by reducing healthcare costs due to hospitalizations, emergency room visits, and facility-based care.

The provided methods and devices build upon and improve prior design methodologies. In prior work, it was shown that LLTE-designed feet can replicate biological function by mimicking able-bodied kinetics and kinematics. See, e.g., Prost, V., Johnson, W. B., Kent, J. A., Major, M. J., and Winter, A. G., 2022. “Biomechanical evaluation over level ground walking of user-specific prosthetic feet designed using the lower leg trajectory error framework”. Scientific Reports, 12(1), pp. 1-15; and Olesnavage, K. M., Prost, V., Johnson, W. B., Major, M. J., and Winter, A. G., 2021. “Experimental Demonstration of the Lower Leg Trajectory Error Framework Using Physiological Data as Inputs”. Journal of Biomechanical Engineering, 143(3), the entire teachings of which are incorporated herein by reference.

Here, it was demonstrated that the LLTE design framework can be used not only to produce experimental prototypes but also to design feet which meet commercial economic, mechanical, and aesthetic requirements. Satisfying these requirements enables a customized prosthetic foot to compete with existing ESR prosthetic feet, which are often made of expensive materials such as carbon fiber or fiberglass. Through a quantitative and predictive design methodology, these objectives can be met with a lower cost but mechanically sound material, Nylon 6/6. Expensive composites are not the only materials which can meet these requirements; when used appropriately, the less expensive Nylon 6/6 can, too.

### EXEMPLIFICATION

### Example 1. Performance Characterization

To demonstrate the ability of the LLTE framework to create amputee-specific prosthetic foot designs using the upgraded parametric foot architecture and constitutive model, prosthetic feet for 27 representative target amputee users with a range of body sizes were designed. The user profiles chosen do not represent the full range of body sizes that could be used as inputs to the LLTE framework. Nevertheless, the range of foot lengths, lower leg lengths, and body masses demonstrates the ability to design personalized feet for patients of significantly different sizes with the provided methodology.

All designs were optimized for level ground walking at a self-selected comfortable walking speed, for a minimum safety factor of at least 1.05 over level ground walking and the ISO ultimate strength loads, and for the properties of Nylon 6/6.

LLTE feet were optimized for the upgraded foot architecture, the results of which are shown in FIG. 7. LLTE scores using the commercial foot architecture were much higher than those obtained using the prior mono-keel, 2-D extruded foot geometry, which were ~0.05-0.06 when designed for users of similar body size and ISO requirements. Despite this difference, all LLTE values are at or below 0.1. It was previously found that prosthetic feet which were designed for level ground walking and have an LLTE score of 0.1 or below facilitate walking performance similar to high end commercially-available carbon fiber ESR prosthetic feet.

With the new parametric foot architecture and upgraded constitutive model, the LLTE framework can be used to design prosthetic feet which fit into the commercial form factor, which includes a split keel, raised heel, and 3-D geometries to fit within a commercial foot shell. This foot form can be quantitatively tuned to design individualized feet for amputees of a range of body sizes, in a personalized and high-resolution way.

### Example 2. Mechanical Validation—Testing of Prototype Foot Stiffness

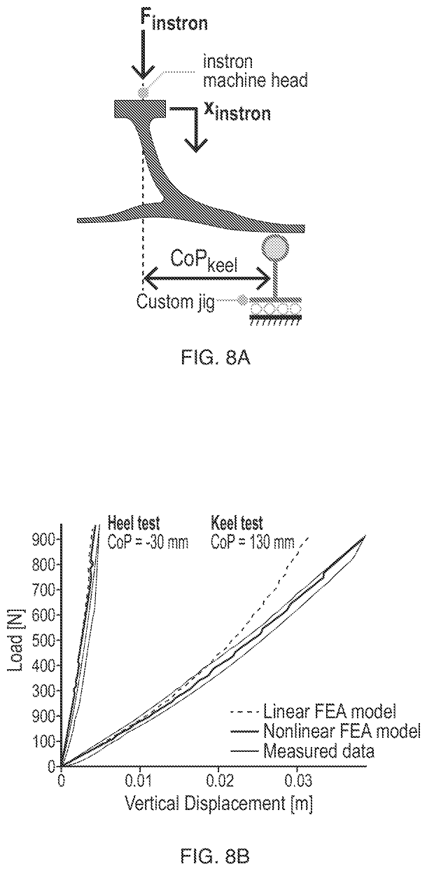

To ensure that the FEA model accurately predicted the foot's deformation and thus its anticipated biomechanical performance, load-displacement curves were measured at representative locations along the bottom of a prototype prosthetic foot. Static mechanical tests were conducted using an Instron load testing machine (Instron Inc., Norwood, MA). The experimental setup consisted of a jig (FIG. 8A), which applied loads to the foot similar to those experienced by the target user when walking on level ground at a self-selected comfortable speed. Loads were applied at representative heel, CoPₕₑₑₗ=−30±0.1 mm and keel, CoPₖₑₑₗ=130±0.1 mm locations, as measured from the prosthesis ankle. The foot was loaded at a constant rate of 300 mm/min to a maximum load F≈1.2 mg, with m equal to the mass of the target user. The vertical load (Finstron) and displacement (xinstron) were recorded at a rate of 50 Hz, with a rated maximum force error of ±6.4% and displacement error of ±0.1 mm in this configuration.

The custom jig included a linear stage on which an aluminum shaft is mounted on a set of roller bearings, which reduces friction and ensures that the applied load remains normal to the bottom of the foot. Adjusting the position of the linear stage changes the location of force application along the bottom of the foot. For each prototype foot, the heel and keel loading used in the Instron testing (load location and magnitude) was replicated using the constitutive model from the LLTE framework, giving model-predicted deformations for the prosthetic feet in each loading condition.

The prototype foot was designed for a representative user with a 26 cm foot size, lower leg length of 0.505 m, and body mass of 78.2 kg. The prototype prosthetic foot was CNC machined from Nylon 6/6 (tensile modulus E=2.49 GPa, tensile yield stress σy=73.7 MPa, poisson ratio ν=0.6, and density ρ=1130 kg/m³). The prototype was tested to a 920 N maximum load five times at both the keel and heel locations.

The constitutive model accurately represented the measured deformations of the prototype prosthetic foot (FIG. 8B). In the heel loading condition, the prosthetic foot's deformation was predicted with an average error of 0.57±0.05 mm relative to a measured average deformation of 4.87±0.06 mm, giving an 11.7±0.9% average error. In the keel loading condition, the constitutive model predicted the foot's deformation with an average error of 0.27±0.18 mm, compared with the average measured deformation of 38.6±0.3 mm for an average error of 0.69±0.48%. The prototype foot also exhibited high energy storage and return efficiency, with an average efficiency of 87.9±3.3%. Frictional losses can be attributed to viscous flow in the material, not to plastic deformation or yielding.

This testing demonstrated that the constitutive model accurately predicted the deformation of the prosthetic foot prototypes. This confirms our ability to quantitatively and predictively design prosthetic feet.

### Example 3. Mechanical Validation-ISO Ultimate Strength Mechanical Testing

To validate that the feet passed the ISO strength tests, as they were designed to, ultimate strength static mechanical tests were conducted on the prototype prosthetic feet. Tests were conducted at both the heel and forefoot, as in ISO 10328, using an Instron load testing machine.

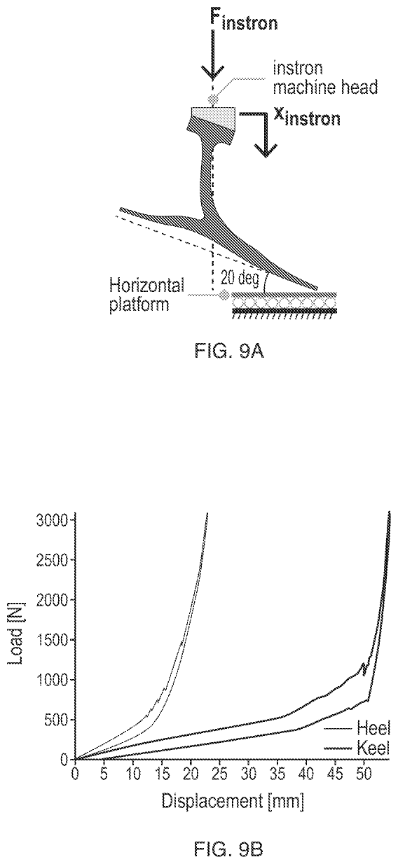

As in prior work, these tests were conducted using a jig which constrained the prototype foot (FIG. 9A) at a prescribed angle while the Instron applied the corresponding ISO ultimate static test loads. Prototype feet were fixed at the required angle (20 deg and −15 deg for the forefoot and heel conditions, respectively) and loaded on a horizontal plate with a teflon (PFTE) sheet, which minimized the shear loading, replicating the effect of the bearing-mounted platform in ISO 10328 [46]. As in the ISO 10328 protocol, feet were loaded at a constant rate of 100 N/s. The vertical load (Finstron) and displacement (xinstron) were recorded at a rate of 25 Hz, with a maximum rated force measurement error of ±2.5% and displacement measurement error of ±0.1 mm. Each test was conducted twice to check for plastic deformation within the foot, which would indicate failure to pass the test.

The prosthetic foot prototype withstood the ultimate static tests on the Instron machine in both the heel and forefoot conditions (FIG. 9B). The foot did not demonstrate signs of failure such as cracks, crazing, or other mechanical failures. The second test resulted in a difference in peak displacement of 0.06 and 0.14 mm for the forefoot and heel loading conditions, respectively. These differences are within the measurement accuracy of the Instron machine, suggesting that the foot underwent minimal, if any, plastic deformation

### Example 4. Mechanical Validation—AOPA Dynamic Heel and Keel Testing

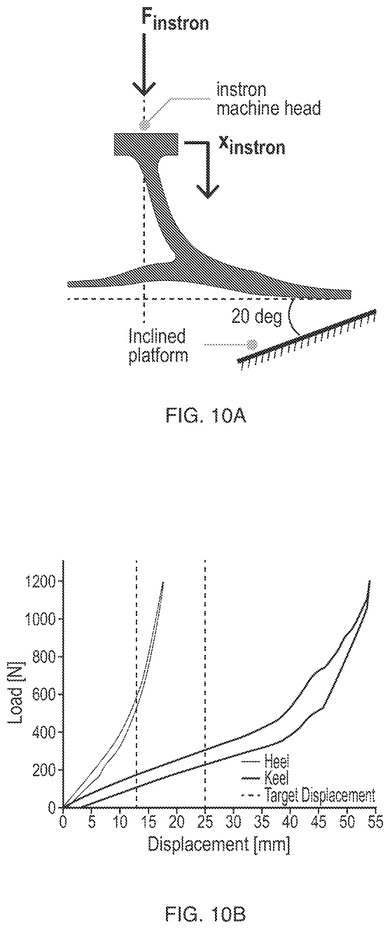

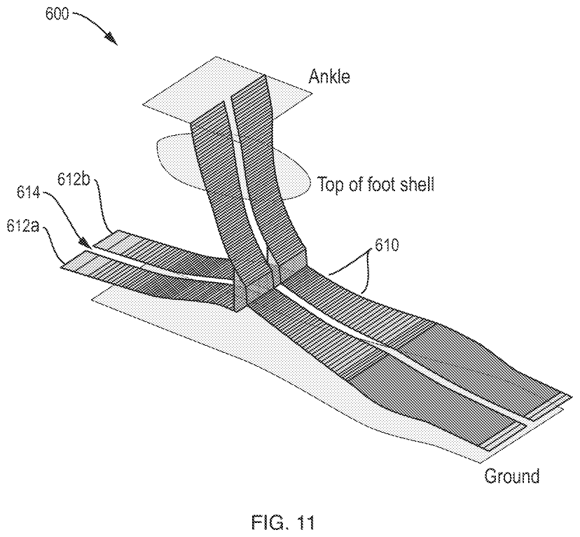

To demonstrate that feet would be classified as dynamic heel, dynamic keel prosthetic feet according to the AOPA test standard, mechanical tests according to the AOPA standards were conducted, using a size 27 cm prosthetic foot designed for a representative 80 kg user. In each test, the prosthetic foot was attached to a pylon, which was rigidly mounted to the Instron cross head (FIG. 10A). The foot was loaded into an aluminum plate, which was mounted to the base of the Instron and rotated to the specified angle (20 deg and 15 deg for the keel and heel conditions, respectively). As in the AOPA dynamic heel and dynamic keel protocol, the foot was loaded at a constant rate of 200 N/s to a maximum load of 1230 N. The vertical load (Finstron) and displacement (xinstron) were recorded at a rate of 25 Hz, with an estimated force measurement error of ±3% and displacement measurement error of ±0.1 mm.

To be classified as having a dynamic keel, a prosthetic foot must deform at least 25 mm under 1230 N, with an energy return efficiency of at least 75% under keel loading. The prosthetic foot deformed an average of 54.2±0.4 mm with an energy return efficiency of 76.4±0.2% (FIG. 9B), which satisfies this standard. For dynamic heel classification, the prosthetic foot must deform at least 13 mm or have an energy return efficiency of at least 82%. The prosthetic foot deformed an average of 17.6±0.04 mm with an efficiency of 88.4±0.1%, passing the dynamic heel standard. By passing both the dynamic keel and dynamic heel mechanical tests, our prosthetic feet satisfy the mechanical criteria, as recommended by AOPA, to be classified as L5981.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of fabricating a compliant prosthetic foot, comprising:
optimizing a set of determinants defining a shape of a compliant prosthetic foot, the shape comprising a keel curve, a heel curve, and a forefoot curve, each of the heel curve and the forefoot curve at least partially defined as an offset curve with respect to a representation of an arched ground-facing surface of the compliant prosthetic foot, the optimizing including minimizing a lower leg trajectory error associated with the set of determinants relative to a target kinematic data set; and
fabricating the compliant prosthetic foot in conformance with the optimized set of determinants.

2. The method of claim 1, wherein the representation of the arched ground-facing surface defines a surface providing for a heel rise and a foot arch for the compliant prosthetic foot.

3. The method of claim 1, wherein the set of determinants of the compliant prosthetic foot (i) comprises at least twelve determinants that include at least one geometric parameter at each of six control points of a parametric curve, or (ii) is set by finite element analysis.

4. The method of claim 3, wherein optimizing the set of determinants comprises optimizing the at least twelve determinants for a prosthetic foot that is compliant along its entire length.

5. The method of claim 1, wherein optimizing the set of determinants comprises at least one of: (i) taking into consideration an intended user's body weight, height, foot size and preferred walking activity, and (ii) a parameterization step for a topology optimization technique.

6. (canceled)

7. (canceled)

8. The method of claim 1, wherein the target kinematic data set is (i) a physiological data set obtained from a subject for whom the compliant prosthetic foot is being fabricated, or (ii) a physiological data set obtained from an able-bodied subject with about the same body size and mass as the subject for whom the compliant prosthetic foot is being fabricated.

9. (canceled)

10. The method of claim 1, wherein the compliant prosthetic foot is fabricated by at least one method selected from the group consisting of: machining; three-dimensional printing; a layup method; a water jet method; additive fabrication; subtractive fabrication; lamination; composite manufacture; injection molding; carbon fiber fabrication; extrusion; casting; molding; co-molding; carving; and vulcanization.

11. The method of claim 1, wherein the compliant prosthetic foot is fabricated of at least one member of the group consisting of: nylon 6/6; carbon fiber; fiber glass; spring steel; titanium; plastic; an alloy of metals; a polymer; a composite; a resin; a thermoset; a thermoplastic; laminate; a rubber; an elastomer; a non-viscoelastic material; a viscoelastic material; and wood.

12. A compliant prosthetic foot fabricated by a process comprising the method of claim 1.

13. A method of fabricating a compliant prosthetic foot, comprising:
optimizing a set of determinants defining a shape of a compliant prosthetic foot, the shape being a two-dimensional shape defined by at least one parametric curve, the optimizing including minimizing a lower leg trajectory error associated with the set of determinants relative to a target kinematic data set;
generating a three-dimensional shape of the compliant prosthetic foot based on the optimized set of determinants, the three-dimensional shape defined at least in part by a set of three-dimensional elements of varying widths; and
fabricating the compliant prosthetic foot in conformance with the generated three-dimensional shape.

14. The method of claim 13, wherein the varying widths of the three-dimensional elements vary over a length of the compliant prosthetic foot to complement an interior region of a foot shell.

15. The method of claim 13, wherein the set of three-dimensional elements comprises paired three-dimensional elements separated by a gap to provide coronal compliance.

16. The method of claim 13, wherein the set of determinants: (i) comprises at least twelve determinants that include at least one geometric parameter at each of six control points of a parametric curve, or (ii) is set by finite element analysis.

17. The method of claim 16, wherein optimizing the set of determinants comprises at least one of: (i) optimizing the at least twelve determinants for a prosthetic foot that is compliant along its entire length, (ii) taking into consideration an intended user's body weight, height, foot size and preferred walking activity, or (iii) a parameterization step for a topology optimization technique.

18. (canceled)

19. (canceled)

20. (canceled)

21. The method of claim 13, wherein the target kinematic data set is a physiological data set obtained (i) from a subject for whom the compliant prosthetic foot is being fabricated, or (ii) from an able-bodied subject with about the same body size and mass as the subject for whom the compliant prosthetic foot is being fabricated.

22. (canceled)

23. The method of claim 13, wherein the compliant prosthetic foot is: (i) fabricated by at least one method selected from the group consisting of: machining; three-dimensional printing; a layup method; a water jet method; additive fabrication; subtractive fabrication; lamination; composite manufacture; injection molding; carbon fiber fabrication; extrusion; casting; molding; co-molding; carving; and vulcanization; or fabricated of at least one member of the group consisting of: nylon 6/6; carbon fiber; fiber glass; spring steel; titanium; plastic; an alloy of metals; a polymer; a composite; a resin; a thermoset; a thermoplastic; laminate; a rubber; an elastomer; a non-viscoelastic material; a viscoelastic material; and wood.

24. (canceled)

25. A compliant prosthetic foot fabricated by a process comprising the method of claim 13.

26. A compliant prosthetic foot comprising:
a keel;
a heel; and
a forefoot, the heel and the forefoot shaped according to a parametric curve at least partially defined as an offset curve with respect to a representation of an arched ground-facing surface of the compliant prosthetic foot.

27. The compliant prosthetic foot of claim 26, wherein the parametric curve is characterized by a set of parameters defining at least three control circles.

28. The compliant prosthetic foot of claim 26, wherein the forefoot and the keel are shaped according to a set of three-dimensional elements of varying widths.
